(19) United States
(12) Patent Application Publication
(10) Pub. No.: US 2001/0002252 A1
(43) Pub. Date: May 31, 2001

(54) TOOTH WHITENING PREPARATIONS

(76) Inventors: ANDREW R. GALLOPO, GARFIELD, NJ (US); NADIR I. IBRAHIM, HACKETTSTOWN, NJ (US); SALVATORE MAZZANOBILE, HAWORTH, NJ (US)

Correspondence Address: WILLIAM T KING, SMITHKLINE BEECHAM CORPORATION, CORPORATE INTEL PROPERTY US UW2220, P O BOX 1539, KING OF PRUSSIA, PA, 194060939

(21) Appl. No.: 08/778,993

(22) Filed: Jan. 6, 1997

Related U.S. Application Data
(63) Continuation of application No. 08/451,798, filed on May 26, 1995, now abandoned.
Application No. 08/451,798 is a continuation of application No. PCT/US94/14662, filed on Dec. 19, 1994.

(51) Int. Cl. A61K 7/16

(52) U.S. Cl. 424/49

(57) ABSTRACT

This invention relates to compositions for whitening teeth and dental prostheses using a water soluble alkali metal tripolyphosphate with or without a source of active oxygen and a enzymatic hydrolytic agent.

### SCOPE OF THE INVENTION

This invention relates to a system for whitening natural teeth and dental prostheses. The whitening is achieved through the use of an alkali metal tripolyphosphate at about between 5 to 25 percent by weight in a dentally acceptable carrier when used alone. The polyphosphate can be combined with a compound which provided active oxygen and/or an enzymatic hydrolytic agent; the polyphosphate may be present in 1 to 25 percent by weight when combined with one or both of these agents. Any orally acceptable presentation, or one suitable for dental prostheses, can be utilized in this invention.

### AREA OF THE INVENTION

Several factors contribute to enamel discoloration but the three main factors are believed to be: i) formation of plaque and tartar matrices on the tooth surface which then entraps stains, ii) ingestion of certain drugs during tooth formation, and iii) discoloration due to oral cavity traumatization following which blood break-down products seep into the mineralized area of the teeth during enamel formation. This invention is primarily concerned with the first factor or cause of tooth discoloration, that is the natural stain which accumulates on teeth.

Over-the-counter teeth whitening preparations have been developed to address the cosmetic preference of many to restore luster to tooth enamel discolored by surface entrapped materials; the term lightening may also be used in conjunction with the advertising and sale of these products. While all dentifrices and mouthwashes contain some cleaning and polishing agents some enamel deposits may become intractable to be fully removed by these agents under normal use conditions. Also these preparations may not be formulated with the amount or type of agent required to fully remove the amount of stains and discoloration which build up due to excessive exposure to the staining agent. For example, smokers often develop discolored enamel because the tars and particulate in exhaled cigarette smoke collect on the teeth. And a number of comestibles can stain or discolor tooth enamel, tea being one example of a beverage where the tannins in the tea deposit on the tooth enamel. Some medicinal agents may cause staining or discoloration via entrapment, though this is not a usual common cause of this type of staining.

Three approaches to enamel whitening are currently in general use. They are based on using abrasives, employ oxidizing agents or utilize a hydrolytic entity to break down the staining material, e.g. enzyme-based products.

One approach is basically a physical abrading of the stain to effect removal. Harsher abrasives, which might also be called polishing agents, than what are normally used in tooth paste preparations are employed in this approach. Most if not all of these preparations are toothpastes, gels or powder dentifrices as they require close contact with the teeth. And brushing or similar scrubbing or polishing action is required as a complement to successful stain removal. Examples of such products are Smokers Topol made by Topol-Dep Corporation and marketed to smokers and tea drinkers as a means for removing stains caused by smoking and drink tea or similar beverages.

Oxidizing agent represent the most widely distributed and utilized agent in oral preparations marketed as enamel whiteners in the U.S. All of these products are pastes or gels. Urea peroxide, hydrogen peroxide or calcium peroxide are most often found in these products. Currently there are more than thirty such products marketed over the counter in the U.S. The oxidizing agents apparently works via the release of a radical which breaks down the plaque/stain complex to a product which can be flushed away or removed by an abrasive. These treatments require quite a bit of time to achieve good results; one-and-a-half to eight days, or 2-3 months depending on the peroxide source and its concentration.

Recently catalytic systems have come back into favor and have been packaged and market through retail outlets in parallel with other oral care products. Proteolytic enzymes are the catalyst of choice, particularly papain. A second active such as a citric acid salt has been used by at least one manufacturer. These products are presented in a paste or gel. They claim to whiten teeth by removing the plaque which has entrapped the stain.

This invention provides a unique alternative. It utilizes a alkali metal tripolyphosphate salt, optionally with a peroxide and/or a hydrolytic agent, to effect stain removal and whiten tooth enamel.

### SUMMARY OF THE INVENTION

This invention comprises a composition for reducing or removing surface deposited stains from natural teeth and dental prostheses comprising a dentally acceptable preparation comprising about 5 to 25% by weight of a water soluble alkali metal tripolyphosphate.

In a second aspect this invention relates to a composition for reducing or removing surface deposited stains from natural teeth and dental prostheses comprising a dentally acceptable preparation comprising about 1 to 25% by weight of a water soluble alkali metal tripolyphosphate, an effective amount of a source of active oxygen, and/or an effective amount of an enzymatic hydrolytic agent.

In addition this invention relates to a means for reducing or removing surface deposited stains from natural teeth and dental prostheses which method comprises contacting teeth or dental prostheses which have surface stains with a dentally acceptable composition comprising at least about 5 to 15% by weight of a water soluble alkali metal tripolyphosphate.

If yet another embodiment, this invention relates to a method for reducing or removing surface deposited stains from teeth and dental prostheses which method comprises contacting said teeth or dental prostheses which have surface stains with a dentally acceptable composition comprising at least about 1 to 25% by weight of a water soluble alkali metal tripolyphosphate and an effective amount of a source of active oxygen, and/or an effective amount of an enzymatic hydrolytic agent.

### DETAILED EMBODIMENTS OF THE INVENTION

The focus of this invention is on that of removing stains which are adhered to, or entrapped in materials on, the surface of teeth or dental prostheses. Native teeth and dental prostheses, e.g., false teeth, can be treated with this invention. This invention can also be used to prevent build-up of surface attached stains. In essence this is a matter of removing periodically small amounts of newly deposited materials, be they stains themselves or matrices which can trap colored materials. Deposit prevention per se may not be involved here but that has not been ruled out. That is, treating teeth with these preparations may prevent the attachment of stains or the entrapment of stains in some fashion. Whether one or both of these phenomena is going on is not so important as the fact that regular use of these preparations can achieve a state where the user does not perceive that her or his teeth are stained. And regular use can prevent a reoccurrence of that condition.

The term stain or staining is used interchangeable with discoloration and generally means that the surface of the enamel (or prostheses) has taken on some unwanted or unnatural coloration distinct from the color of the underlying enamel. These words are intended to be given the same meaning as here as would be accorded to them in their contemporary usage in the oral and dental care arts.

These preparations and the methods disclosed herein are directed toward human use though these preparations can be used in other species as well, for example in pet care products.

The primary active component of this invention comprises a water soluble alkali metal tripolyphosphate. The sodium form of this salt is preferred, though the potassium or mixed sodium and potassium salts could be used as a preferred embodiment as well. All physical forms can be used, e.g. a hydrate or the dehydrated form.

The amount of tripolyphosphate salt will be between about 5 and 15% by weight of the preparation when it is used alone in a solid preparation, that is neither of the other two actives are present in the defined ranges. A solid preparation includes a paste, gel, powder and the like. A preferred amount of said salt, again when used alone in a solid preparation, is about 10% by weight/weight.

As regards liquid preparations, e.g., mouthwashes or rinses, the tripolyphosphate can be used in amounts ranging between 0.1 to 15% by weight/volume. A more preferred range is 1 to about 10% by weight/volume in a mouthwash or rinse.

When a peroxy compound or a hydrolytic enzyme is added in with the tripolyphoshpate in a solid preparation, or a liquid preparation such as a mouthwash, the phosphate may be used in amounts ranging between 1 and 25% by weight/weight in solid preparations and in a range of about 0.1 to 15% weight/volume in liquid preparations. A preferred amount of the phosphate in the multi-component solid preparations is between 5 and 10% (w/w), most preferably about 10%.

A source of active oxygen means material containing an O-O bond which can break down to give an active oxygen specie(s). The peroxides and hydroperoxides are preferred for use in this invention. Examples of preferred peroxides are inorganic peroxides such as hydrogen peroxide, the alkali metal percarbonates such as sodium, potassium and calcium percarbonate; sodium, calcium, strontium, bailum, zinc or magnesium peroxide, and the perborates. Also inorganic compounds which contain hydrogen peroxide of crystallization, such as 2 Na₂CO₃.3H₂O₂ and Na₄P₂O₇.nH₂O₂ can be used. In addition several organic peroxides can be used herein. For example peroxy compounds such as peracetic acid, benzoyl peroxide, magnesium monoperphthalate, m-perchlorobenzoic acid and cumyl hydroperoxide can be used in this invention. This is a representative list and is not intended to limit in any way or fashion the practice of this invention vis-a-vis what comprises an active oxygen compound.

The concentration of these active oxygen compounds (AOCs), that is the effective amount, will vary between about 0.01% and 20% in the final form of the composition as measured just before it is contacted with the teeth or prosthesis. These percentages may be calculated on the basis of weight where the AOC is a solid and the dentifrice is a solid. If the AOC is a liquid and the dentifrice a solid, then the percentages will be based on a weight/weight calculation. Likewise where the AOC is a solid and is being added to a liquid, i.e., a mouthwash, the percentages will be based on a weight and volume calculation. If the AOC and the final product are both liquids, then the percentage will be based on a volume/volume calculation. For convenience herein, recitation of a percentage for an AOC should be read to include all four of these instances; it will be left to the practitioner to select the appropriate calculation based on what preparation she is formulating.

Enzymes which break down proteins, lipids or sugars can be added to these preparations. Herein the phrase “enzymatic hydrolytic agent” is used as a short-handed means for referring to these types of enzymes. These enzymes are well known in the art of enzymology. They are usually referred to as proteases, lipases and amylases respectively. Numerous papers and reviews have been written on the activity of these enzymes and their physical and biological characteristics. See for example “Advances in Enzymology and Related Subjects,” John Wiley & Sons, Inc. all editions and Methods in Enzymology, Academic Press, New York, all editions.

The hydrolytic agent may comprise a single enzyme or a mixture of a given type of enzyme or still further a mixture of two or three types of enzymes. It is preferred to use a protease in singular combination with the other active(s). And it is most preferred to use just one protease. But an amylase or a lipase could be combined with the protease, or both could be added in with the protease, to provide a means for enzymatically degrading proteins, fats and sugars.

All hydrolytic agents may be combined with the tripolyphosphate with no adverse affect on the activity of the agents or on the activity of the phosphate per se. Certain formulation parameters, such as pH, may affect the activity of a given enzyme. It is well known that enzyme activity is influenced by pH. For example some enzymes are more active at a pH below 7, some are most active at a neutral pH and others are most active at a pH higher than 7. Formulation with a particular enzyme must necessarily take into consideration the pH at which the target enzyme is most active. And in addition, the pH of the product during use may have an influence on selecting an enzyme. Certain of the alkali metal tripolyphosphates which can be used in this invention are most stable at an alkaline pH. So it is preferred to use an enzyme which retains its activity when exposed to alkaline pH, or to protect the enzyme in some fashion such as encapsulating it or presenting it in a separate container for combination with the phosphate at time of use.

Certain enzymes which require a heavy metal ion to function may not be particularly useful in this invention given the presence of the tripolyphosphate. But it may be possible to supplement the preparation with that ion, thereby providing it in concentrations adequate to maintain the activity of the enzyme while not adversely impacting the action of the tripolyphosphate on stains.

In another embodiment, certain enzymes may be combined with the active oxygen component to provide a dentifrice with three actives. Oxidizing agents such as peroxides degrade enzyme, inactivate them, rather rapidly. But it has been found that the rate of degradation for certain enzymes is not so rapid that they lose all hydrolytic effectiveness immediately, e.g. in less than a minute. And it has been found that this residual activity is sufficiently high to provide for protein and other stain hydrolysis over five to ten minutes. Since the methods of this invention will normally be carried out over one to ten minutes, the invention also contemplates a tripartite preparation combining the tripolyphosphate, a source of active oxygen and a hydrolytic agent which retains useful activity within this time frame. These relatively stable enzymes are termed peroxide-active enzymes herein.

A peroxide-active enzyme is any enzyme having measurable activity at 3% (w/v) hydrogen peroxide in aqueous solution at standard temperature and pressure as determined by such colorimetric assays as the Azocoll method, Tomarelli, R. M., et al., J. Lab. Clin. Med., 34, 428 (1949), or the dimethyl casein method for determining proteolytic activity as described by Yaun Lin, et al., J. Biol. Chem., 244: (4) 789-793, (1969).

These stable enzymes may be derived from any plant or animal source, including microbial and mammalian sources. They may be neutral, acidic or alkaline enzymes.

A proteolytic enzyme will have in part or in total the capacity to hydrolyze peptide amide bonds. Such enzymes may also have some inherent lipolytic and/or amylolytic activity associated with the proteolytic activity.

Preferred proteolytic enzymes are those which are substantially free of sulfhydryl groups or disulfide bonds, whose presence may react with the active oxygen to the detriment of both the activity of the active oxygen and which may result in the untimely inactivation of the enzyme. Metallo-proteases, those enzymes which contain a divalent metal ion such as calcium, magnesium or zinc bound to the protein, may also be used, with the foregoing caveat that the phosphate may have an affect on the activity of such enzymes.

A more preferred group of peroxide-stable proteolytic enzymes are the serine proteases, particularly those derived from Bacillus and Streptomyces bacteria and Asperigillus molds. Within this grouping, the more preferred enzymes are the Bacillus derived alkaline proteases generically called subtilisin enzymes.

Reference is made to Deayl, L., Moser, P. W. and Wildi, B. S., “Proteases of the Genus Bacillus. II alkaline Proteases.” Biotechnology and Bioengineering, Vol. XII, pp 213-249 (1970) and Keay, L. and Moser, P. W., “Differentiation of Alkaline Proteases from Bacillus Species” Biochemical and Biophysical Research Comm., Vol 34, No. 5, pp 600-604, (1969).

The subtilisin enzymes are broken down into two sub-classes, subtilisin A and subtilisin B. In the subtilisin A grouping are enzymes derived from such species are B. subtilis, B. licheniformis and B. pumilis. Organisms in this sub-class produce little or no neutral protease or amylase. The subtilisin B sub-class is made up of enzymes from such organisms as B. subtilis, B. subtilis var. amylosacchariticus, B. amyvoliquefaciens and B. subtilis NRRL B341 1. These organisms produce neutral proteases and amylases on a level about comparable to all their alkaline protease production.

In addition other preferred enzymes are, for example, pancreatin, trypsin, collaginase, keratinase, carboxylase, aminopeptidase, elastase, and aspergillo-peptidase A and B, pronase E (from S. griseus) and dispase (from Bacillus polymyxa).

Preferred proteases are papain, bromelin, and the various subtilisins mentioned above.

The identification, separation and purification of enzymes is an old art. Many identification and isolation techniques exist in the general scientific literature for the isolation of enzymes, including those enzymes having proteolytic and mixed proteolytic/amylolytic or proteolytic/lipolytic activity. The enzymes contemplated by this invention can be readily obtained by known techniques from plant, animal or microbial sources.

With the advent or recombinant DNA techniques, it is anticipated that new sources and types of enzymes will become available. These newly minted enzymes should be considered to fall within the scope of this invention so long as they meet the criteria for stability and activity set forth herein.

An effective amount of enzyme is to be used in the practice of this invention. The amount of enzyme required to make an effective dentifrice will depend on several factors, including the inherent activity of the enzyme, pH, salt concentration, and if combined with a peroxide, its susceptibility to attack by peroxides.

As a basic yardstick, the working formulation should contain sufficient enzyme to provide between about 0.001 to 5 Anson units of activity, preferably between about 0.01 and 1 Anson units, per single treatment. Higher or lower amounts may be used. Enzyme concentrations lower than these stated here probably will serve to assist in removing surface materials which stain, particularly if used to clean prostheses which can be removed from the mouth and soaked for some extended period such as overnight. In weight/weight or weight/volume terms, since enzyme preparations are seldom pure, it is expected that the enzyme source will be used in amounts between about 0.003 to 15% of the final working preparation. The precise amount will vary with the purity of the enzyme and will need to be finally determined on a lot-by-lot basis.

These preparations will be presented in a form which is safe for use in the oral cavity and which will not have a deleterious effect if accidentally swallowed. The oral care art has developed a substantial body of formulation types and has identified and tested a large listing of ingredients which can be used in these preparations in a safe and efficacious manner. Confecting or manufacturing these preparations, and there safe packaging and storage is also well documented in these arts.

For achieving optimum whitening results the formulation as used should have a pH of between about 7.0 to 9.0. Optimum cleaning results are achieved with a pH of about 8 with reference to how the formulation is used for brushing teeth in the mouth. For example a toothpaste preparation will usually be diluted in the mouth by about 1 plus 2 or 1 plus 3 volumes of water/saliva while brushing. Thus a paste or gel, for example, optimized for whitening will be formulated to achieve a pH between about 7 and 9 when it is being actually used to brush or treat teeth. An acid or base may be used to adjust the pH of the preparation; the choice is within the skill of the art concerned with oral preparations. The optimum pH for stability purposes in a particular preparation, prior to use, may vary from this range and may have a different optimum pH, depending on excipients and additives.

While the preparations can be presented in any form, it is contemplated that certain embodiments, as compared with ingredient mixes, will provide better and faster results, and will be more user friendly. By example, one can provide preparations in the form of an oral rinse, a gum, a dentifrice such as a paste, gel or powder, or one can impregnate the active into a toothpick or dental floss. Appliqués and mouth pieces can also be used, that is preparations which are applied to the teeth and left in place for several hours with or without a covering membrane to secure them in place. While the method can be practiced in a professional setting, it is intended primarily in a self-treatment regimen.

In addition, a course of care may include using two or more of these presentations. Say for example a mouthwash can be used in conjunction with a tooth paste where both contained the two ingredients. Such a regimen can contribute to optimizing the effect of this oral care regimen, particularly in those consumers who normally use both products. Flossing in conjunction with using tooth paste can also comprise an effective treatment regimen.

It is contemplated that the preparations used in this invention would be formulated in a way which permitted them to be used in the fashion and for the time the consumer would normally associate with the use of that presentation. For example, if the actives were presented as a mouth wash, it would be formulated so that the directions for use would be consonant with the normal and accepted practice of using mouth washes. The same applies for dentifrices, toothpicks, or dental floss and chewing or bubble gums. No special time requirements are contemplated for the practice of this invention. If an applique or mouth piece approach is used, that necessarily entails keeping the materials on the teeth for up to several hours, or more. That is the only context where extended treatment is the norm.

Preferably the active will be formulated into a tooth paste, liquid paste or gel. So for example 10% sodium tripolyphosphate (STP) will be mixed with other orally acceptable ingredients to make a paste or gel which will be packaged in a usual fashion. The consumer will then put some of this gel or paste on a tooth brush and brush her teeth for a discrete time, just as if she was using any other tooth paste. Most dentists and researchers recommend brushing ones teeth for at least three minutes per brushing to achieve maximum results, though compliance with this standard is not universal. A similar standard is recommended for the instant pastes and gels, though it is expected that non-compliance will still provide the desired results with regular use, i.e. daily use.

Any orally acceptable carrier or carriers can be used, with the limitation that multivalent metal ions should be eliminated or minimized, otherwise these ions will form a complex with the active and could prematurely render it partially or wholly ineffective. In addition, oxidizing agents should not be used lest they react with the reducing agent. Otherwise, the two actives can be formulated with any compatible excipients which are also acceptable for use in the oral cavity.

Oral Preparations

Dentifrices

Foundation formulations for toothpastes, liquid pastes, gels and toothpowders which can be used in this inventions will have the usual carriers, binders, surfactants, humectants, coloring agents, pigments, antiplaque agents, anti-bacterial agents, bioadhesive-type agents, abrasives, anticaries agents, flavorings, sweeteners, bulking agents and the like which can be used in preparing pastes and powders. Gels and pastes contain water or can be anhydrous.

Dental abrasives are useful with these actives as a means for providing physical removal of materials which have been acted on by the phosphate. Classic examples of dental abrasives are calcium pyrophosphate, silica abrasives, alumina, insoluble metaphosphates, particulate thermosetting polymerized resins, and sodium bicarbonate. The patent and scientific literature is replete with examples of such abrasives. One such example is U.S. Pat. No. 4,822,599 which list a series of dentifrice abrasives and references commercial sources and literature references on their preparation. Most if not all of dental abrasives are available from commercial sources.

A selected abrasive should be compatible with the phosphate active, as well as any additives which may be actives in their own right, such as fluoride ions and antibacterial agents. But as with any other paste, gel or powder, the selection of an abrasive can be influenced by the consequence of combining a particular abrasive with another additive. For example if fluoride ions and calcium pyrophosphate ions are to be included in these preparations the pyrophosphate should be converted from its γ-phase to its β-phase by heating the γ-phase to 700°-900° C. as per the teachings of U.S. Pat. No. 3,112,247. Also certain quaternary ammonium-based antibacterial agents may not be compatible with some silica abrasives. Silica is a preferred abrasive for this work.

Abrasive concentrations can cover a very broad range. Preparations are described with abrasive ranging in concentration from 5 to 80% by weight depending on the abrasive. A secondary concentration range is that of 10 to 50% depending on the abrasive selected. Herein the preferred abrasive, silica, is employed in amounts between 10 and 20% by weight.

A source of fluoride ion may be included in these preparations. Fluoride ion sources are numerous. For example see U.S. Pat. No. 3,535,421 which lists numerous salts which can be used in the dental arts. While any one of these sources could be used sodium fluoride, stannous fluoride and sodium monofluorophosphate have emerged as the preferred ion sources in most dentifrices.

Fluoride ions are routinely added into dentifrices in an amount sufficient to provide up to 3500 ppm, preferably 1100 ppm of the fluoride ion. Where a preparation is formulated such that the fluoride ion is confined to one component of the preparation, but is mixed with the other components at the time of use, the fluoride ion source should be adjusted upward in an amount sufficient to provide a concentration of up to about 3500 ppm, but preferably 1100 ppm, in the product as used.

As for other components, flavorings, coloring agents, sweeteners, humectants, thickening agents, binders and surfactants are most commonly used in dentifrices.

Taste is provided by adding a small amount of a flavoring agent; this component also leaves a perception of mouth freshness. Numerous minty flavored agents are available for use in dentifrices and it is well known in the art how to go about selecting a flavoring and testing its consumer acceptability. Flavoring agents are routinely used at levels of between about 0.1 to 5% by weight.

Dyes, lakes and titanium dioxide are routinely used in the dentifrice arts for imparting color; in the case of titanium dioxide a white paste or powder is obtained. These materials are widely available, have been oft used in such formulations and are well known to the dental artisan. Coloring agents are usually present in concentrations ranging between 01. and 5%.

Sweeteners are routinely added to increase consumer acceptability. So called artificial sweeteners are used today to avoid the cariogenic potential of most sugars and other sweetening agents. Examples of non-cariogenic sweeteners now in routine use are saccharin, aspartame, D-tryptophan, dihydrochalcones, cyclamates, xylitol and acesulfame. Sweeteners comprise about 0.1 to 5% of the formulation.

Humectants are added to gels and pastes to prevent their drying out on exposure to air, and they impart a “moist” feel to the mouth when brushing. Some humectant, e.g. sorbitol, are perceived as sweet. Examples of compounds useful as humectants in dentifrices are the polyhydric alcohols such as glycerin, sorbitol, and polyethylene glycols. Sorbitol (usually 70% sorbitol/water) and glycerin are preferred. In pastes and gels one or two humectants are usually used in amounts between about 20 and 40%.

Binders and thickening agents can be added to assure physical integrity in pastes, gels and liquid pastes. Examples of these are gums such as xanthan and acacia gum, carageenan, the celluloses such as carboxy methyl cellulose, polyoxyalkyl polymers such as the Pluronics polymers, PVP materials, and certain polymers exemplified by the carboxyvinyl polymers (Gantrez and the like). These latter polymers, and perhaps some of the others, have an additional benefit in that their adhesive nature which makes them useful as binders also can serve the additional purpose of adhering to the teeth surface and thereby binding the active ingredients to the teeth for a longer period. Gantrez is a example of a polyacrylic carboxylate material which serves such a dual purpose.

Binders are usually added in amounts ranging between 0.1 and 5.5 by weight.

Surfactants normally are added to dentifrices to assist with removing debris. All classes of surfactants, anionics, cationics, amphoterics, nonionics and zwitterion-based surfactants, can be used in these preparations. These compounds, and those which are most useful in the dental arts, are well documented in the literature. Reference is made to U.S. Pat. No. 4,822,599 for a detailed listing of useful surfactants. Surfactants are available through any number of commercial manufacturers or can be make by well documented processes.

Surfactants are normally used in amounts between about 0.5 and 5% in pastes and gels but may be used at higher concentrations in some dental powders. Surfactants can also be used as gelling agents.

Breath freshening agents (eg. sodium bicarbonate) could be included, either with the phosphate preparation or as an adjunct a described in the preceding paragraph, i.e. a two chamber delivery device.

Appliqués can provide an effective means for removing stains as per this invention. These can be prepared in the form of a doughy or tacky material which can be readily molded to conform to the teeth. It can then be manually compressed on the teeth as is or placed in a plastic retainer, inserted into the mouth and bitten into, and left in place of a some time, for example 15 to 30 minutes. When the appliqué is removed the debris causing the stain will be removed. The appliqué is then discarded.

The active(s) can be formulated as a mouthwash or mouth rinse as well. A mouth wash or rinse will contain up to 95% water, up to 30 % alcohol, flavor, polyhydric alcohols, anti-caries agents, plaque removing agents, sweeteners, dyes and lakes, and a preservative in some instances, and sufficient water to make volume. The active could also be incorporated into currently existing formulations such as Cepacol (Lakeside Pharmaceuticals), Plax, (Pfizer), Listerine (Warner-Lambert), Scope (Procter & Gamble), and the like.

Concentrations of the active phosphate in these products would be in the range of about 1 to 15% (weight/volume), preferably 1 to 5%. An AOC could be mixed with the phosphate, or presented in a separate dispenser for combining with the phosphate-containing preparation at time of use. Likewise, an enzyme could be mixed with the phosphate or package separately for mixing with the phosphate at time of use. If all three ingredients are to be presented in a mouthwash or rinse, it is contemplated that the enzyme will be packaged separately. Any one of these ingredients maybe packaged as a powder for mixing and then diluted up in a liquid containing the other ingredient(s) just before use.

A soaking and cleaning solution for dental pieces can also be prepared with these two active ingredients. It is contemplated that such preparations would contain water, a surfactant, an effervescing agent, and other optional ingredients. Dental prostheses would be removed and placed in a solution containing the tripolyphosphate salt and soaked for several hours, then either brushed with a recommended dentifrice or simply rinsed and reinserted into the mouth. The concentration of phosphate active here will be about 1-20%.

The following examples are provided by way of illustration and are not intended to limit the scope of the invention.

### EXAMPLE 1

### Formulation of Tooth Whitening Toothpaste

A toothpaste representative of what may be prepared for the practice of this invention was prepared as per the ingredient profile and percentages in Tables I and II.

TABLE I
(10/15% STP)

| Ingredient | Concentration (W/W %) | |
|---|---|---|
| Water, DI | 28.402 | 23.402 |
| Sorbitol, 70% | 26.410 | 26.410 |
| Abrasive silica | 14.00 | 14.00 |
| Sodium Tripolyphosphate (STP) | 10.00 | 15.00 |
| Glycerin | 10.000 | 10.000 |
| Thickening silica | 4.00 | 4.00 |
| Polyethylene glycol 400, NF | 3.00 | 3.00 |
| Sodium Lauryl Sulfate | 1.150 | 1.150 |
| Titanium Dioxide | 0.995 | 0.995 |
| Sodium Saccarhin | 0.20 | 0.20 |
| Xanthan Gum | 0.800 | 0.800 |
| flavor | 0.800 | 0.800 |
| Sodium Fluoride | 0.243 | 0.243 |
| Total | 100.00 | 100.00 |

TABLE II
(10% STP and Hydrogen Peroxide)

| Ingredient | Part A | Part B |
|---|---|---|
| Sobitol Solution (USP 70%) | 23.0901 | — |
| Glycerin 99.5%, USP | 10.000 | 88.00 |
| PEG 8 | 3.00 | — |
| Silicon Dioxide, (Zeofree 153) | 4.500 | — |
| Silicon Dioxide, (Zeodent 113) | 10.000 | — |
| Titanium Dioxide | 1.447 | — |
| Sodium Lauryl Sulfate NF | 1.150 | — |
| Saccharin, Sodium Powder USP | 0.40 | — |
| Sodium benzoate, NF | 0.100 | — |
| Xanthan Gum | 0.700 | — |
| Sodium Tripoly phosphate | 20.000 | — |
| Sodium Hydroxide 50% solution | 0.900 | — |
| Carbopol 974P | — | 2.000 |
| Hydrogen Peroxide (35%) | — | 10.000 |
| Flavor (not to exceed) | 1.5 | |
| Water | QS | |
| Total | 100.00 | 100.00 |

These two formulations will be packages in containers which dispense an equal volume of each; the 1to-1 ratio of Part A and Part B comprise the product to be used for brushing the teeth or dental prostheses.

TABLE III
(5% STP optionally with papain)

| Ingredient | Concentration (W/W %) | |
|---|---|---|
| Water, DI | 28.402 | 28.302 |
| Sorbitol, 70% | 31.410 | 31.410 |
| Abrasive silica | 14.00 | 14.00 |
| Sodium Tripolyphosphate (STP) | 5.00 | 5.00 |
| Glycerin | 10.000 | 10.000 |
| Thickening silica | 4.00 | 4.00 |
| Polyethylene glycol 400, NF | 3.00 | 3.00 |
| Sodium Lauryl Sulfate | 1.150 | 1.150 |
| Titanium Dioxide | 0.995 | 0.995 |
| Sodium Saccarhin | 0.200 | 0.200 |
| Xanthan Gum | 0.800 | 0.800 |
| Flavor | 0.800 | 0.800 |
| Sodium Fluoride | 0.243 | 0.243 |
| Papain | | 0.100 |
| Total | 100.00 | 100.00 |

### EXAMPLE 2

Tooth whitening was measured by the following process:

Whitening was determined using a modification to a system normally employed in the dental arts for determining the abrasivity of dentifrices.

Bovine jaws were obtained at a local abattoir and the teeth were extracted in their native state; no pre-cleaning was done. Teeth were mounted in a cap with dental acrylic so as to fit the format of a colorimeter and the brushing instrument: a detailed description is given below. An initial brushing was done with a slurry of Crest Regular toothpaste and an Oral B40, medium bristle, toothbrush. Then an initial L value was determined on a colorimeter (Hunter). This was designated as the initial whiteness value. The wear tester device was manufactured by Ramè-Hart, of Mountain Lakes, N.J. This machine has often been used to test the abrasivity of dental preparations. Tooth brushes were mounted and the teeth and brushes aligned. A 1:3 slurry of paste and water was poured into the trays and brushing commenced for a total of six hours. The slurry was replaced hourly. At 3 and 6 hours L value determinations were made. This was done by first rinsing the teeth with deionized water, then placing them in a closed container at 100% humidity for 1 hours, then taking a reading on the colorimeter to obtain a 3 hour and 6 hour L value.

Toothpaste slurries were tested for their whitening effect. An exemplary slurry was prepared as follows:

The placebo was prepared using the ingredients listed in Table I, but without any STP. To a portion of this was added STP, either 5, 10 or 15 percent, and the STP and the base paste were mixed till all were combined. The pH of this preparation, that is with the STP, was approximately 8.5. Where higher pH slurries were prepared, 3M NaOH was used to adjust the pH and where the pH was to be lowered, 3M HCl was used. All slurries were used immediately. The results showed that 10% or higher STP provided superior tooth whitening as compared with the placebo base and that the best results were obtained at a pH of around 8.0.

What is claimed is:

1. A composition for reducing or removing surface deposited stains from natural teeth and dental prostheses comprising a dentally acceptable preparation comprising about 5 to 15% by weight of a water soluble alkali metal tripolyphosphate.

2. A composition for reducing or removing surface deposited stains from natural teeth and dental prostheses comprising a dentally acceptable preparation comprising about 1 to 25% by weight of a water soluble alkali metal tripolyphosphate, an effective amount of a source of active oxygen, and/or an effective amount of an enzymatic hydrolytic agent.

3. A method for reducing or removing surface deposited stains from natural teeth and dental prostheses which method comprises contacting teeth or dental prostheses which have surface stains with a dentally acceptable composition comprising at least about 5 to 15% by weight of a water soluble alkali metal tripolyphosphate.

4. A method for reducing or removing surface deposited stains from teeth and dental prostheses which method comprises contacting said teeth or dental prostheses which have surface stains with a dentally acceptable composition comprising at least about 1 to 25% by weight of a water soluble alkali metal tripolyphosphate and an effective amount of a source of active oxygen, and/or an effective amount of an enzymatic hydrolytic agent.
